(19) United States
(12) Patent Application Publication
(10) Pub. No.: US 2019/0380636 A1
(43) Pub. Date: Dec. 19, 2019

(54) Vein Corral

(71) Applicants: Lisa Nixon, Clarkston, WA (US); Clint Olson, Clarkston, WA (US)

(72) Inventors: Lisa Nixon, Clarkston, WA (US); Clint Olson, Clarkston, WA (US)

(21) Appl. No.: 16/009,273

(22) Filed: Jun. 15, 2018

(51) Int. Cl. A61B 5/15; A61M 5/42

(52) U.S. Cl. CPC A61B 5/150748; A61M 5/427

(57) ABSTRACT

This device makes accessing a vein practically fool-proof. The long, slender tines corral the vein, holding it snuggly in place and prevents the vein from ‘rolling’. This will reduce the number of missed attempts and reduce the number of needle sticks to the patient. The slight arc of the apparatus allows the health care professional temporarily occlude the vein in lieu of a tourniquet by simply rocking the device back and applying pressure. By rocking the device forward and reducing the pressure blood is allowed to flow free again.

### FIELD OF THE INVENTION

The present invention relates generally to an apparatus for a medical device. Specifically, the present invention is an apparatus for a medial device that allows the user to firmly grip a vein in preparation for medical procedures like I.V. starts and blood draws.

### BACKGROUND OF THE INVENTION

Finding and then handling a vein while performing a medical procedure can be difficult and may make an already uncomfortable process, for some, even worse. It can also be very frustrating for the medical professional. As an R.N. this inventor has tested the item and it works. It will be a medical supply item as common as tongue depressors, cotton swabs and safety needles.

### BRIEF SUMMARY OF THE INVENTION

By corralling the vein between the fork like tines it stops the vein from rolling or moving while the medical professional is attempting to access the vein for I.V. starts or blood draws. Thus practically eliminating a missed attempt requiring multiple pokes with a needle. This will reduce the anxiety and frustration of patient and medical professional when it comes to accessing a vein.

### BRIEF DESCRIPTION OF THE DRAWINGS/PHOTOS

All illustrations are for the purpose of describing selected versions of the present invention and are not intended to limit the scope of the present invention.

FIG. 1 is a picture of the present invention;

FIG. 2 is a picture of the present invention;

FIG. 3 is a drawing of the present invention;

FIG. 4 is a drawing of the present invention.

### DETAIL DESCRIPTION OF THE INVENTION

The preferred embodiment of the present invention may be referred to hereinafter as a “Vein Corral”. The Vein Corral is a ‘fork’ like item that is meant to ‘corral’ a vein and prevent it's moving or rolling during an access procedure such as an I.V. or a blood draw. It holds the vein in place while inserting a hypodermic needle.

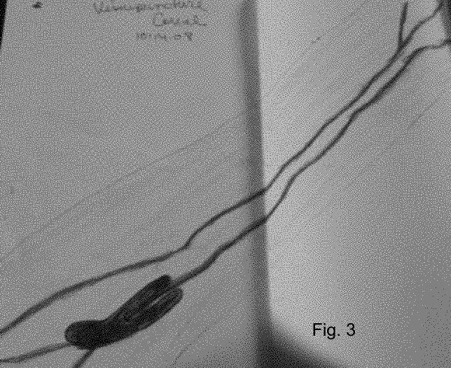

The apparatus has an elongated body which in a side view circumscribes a partial arc of a circle (approximately 5-15 degrees of an arc, depending on the size of the apparatus). This is shown in FIG. 3.

The device is generally between 3 and 4½ inches long and 1½ to 2½ inches wide, depending on the size used. Pediatric patients would obviously require the smallest size while an adult could require different sizes from small to extra large.

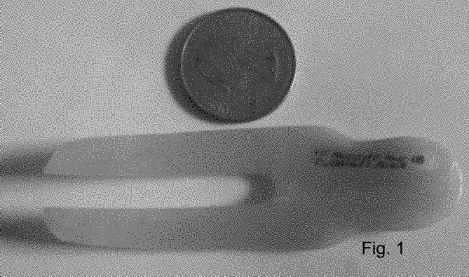

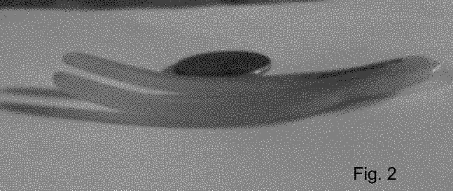

In a plan view, the body is defined by an elongated slot which is open at one end and the opposite end of the slot is closed. The slot thus defines two essential parallel tines, or a fork in the body. The closed end of the device has a small ‘button’ type handle. This is shown in FIGS. 1 and 2.

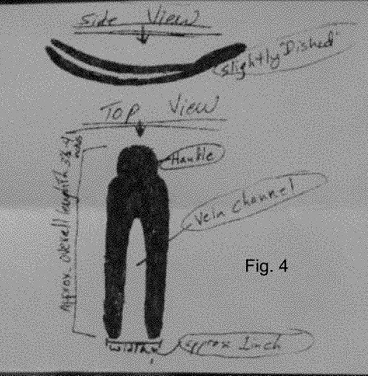

A vein is ‘corralled’ in the slot by the tines when the apparatus is pressed flat against the arm (or other area of the body) over and in line with the vein to be accessed. While holding the handle and applying more pressure a vein can be temporarily occluded in lieu of a tourniquet and then ‘rocked’ forward allowing the blood to flow freely. This is the reason the device is slightly arched. Shown in FIG. 4.

The device is generally rigid, but can have some slight resiliency to it. Materials of manufacture include plastics, metal or, polymers and the like. Plastics are preferred as to manufacture as a sterile, disposable product.

The appliance is used to corral a vein in preparation for venous access for starting an I.V. or drawing blood. It is made to be disposable (1× only use) and in various sizes. It can be used in all areas of medical practice from E.R., surgery, labs, Dr. offices, hospitals, pediatrics, etc., anywhere access to a vein is required.

The tines (fork) corral and hold the vein, the arch (dish shape) allows the user to apply pressure to occlude the blood flow and release the pressure to allow the blood to flow freely again. Also, allows for pressure to placed to isolate and corral the vein without occlusion of the blood flow until occlusion is needed. User simply rocks the device back or forward to block or release blood flow. This may eliminate the need for tourniquet use for most patients.

Although the invention has been explained in relation to it's preferred embodiment, it is to be understood that many other possible modifications and variations can be made without departing from the spirit and scope of the invention.

1. This device prevents movement or ‘rolling’ of the vein to be accessed for I.V. starts or blood draws, allowing easier access to the vein which significantly reduces missed attempts leading fewer needle sticks. (Get it right the first time).

2. Using this device reduces anxiety of the patient and the frustration of the health care professional by increasing the ease of access to the vein and decreasing the need for multiple pokes with a needle.

3. Because this device makes access to a vein so much easier it will increase the number of nurses/health care professionals that can actually start an I.V. or draw blood.
